(19) United States
(12) Patent Application Publication
(10) Pub. No.: US 2010/0291690 A1
(43) Pub. Date: Nov. 18, 2010

(54) METHODS FOR DETECTING OR MONITORING CANCER USING LPC AS A MARKER

(76) Inventors: Lian Shan, Broadview Heights, OH (US); Rebecca Sutphen, Tampa, FL (US)

Correspondence Address: HOLTZ, HOLTZ, GOODMAN & CHICK PC, 220 Fifth Avenue, 16TH Floor, NEW YORK, NY, 10001-7708, US

(21) Appl. No.: 12/734,117

(22) PCT Filed: Nov. 5, 2008
(86) PCT No.: PCT/US08/12483

§ 371 Date: Apr. 19, 2010

Related U.S. Application Data
(60) Provisional application No. 61/002,282, filed on Nov. 7, 2007.
(60) Provisional application No. 61/002,989, filed on Nov. 14, 2007.
(60) Provisional application No. 61/006,331, filed on Jan. 7, 2008.

(51) Int. Cl. G01N 33/487

(52) U.S. Cl. 436/64

(57) ABSTRACT

A method of detecting a cancer, such as ovarian cancer, in a test subject including (a) determining the amount of a lysophosphatidyl choline in a sample of a bodily fluid taken from the test subject, and (b) comparing the amount of the lysophosphatidyl choline in the sample of the bodily fluid taken from the test subject to a range of amounts of lysophosphatidyl choline found in samples of the bodily fluid taken from a group of normal subjects of the same species as the test subject and lacking the cancer, such as ovarian cancer, whereby a change in the amount of the lysophosphatidyl choline in the sample of the bodily fluid taken from the test subject indicates the presence of the cancer, such as ovarian cancer.

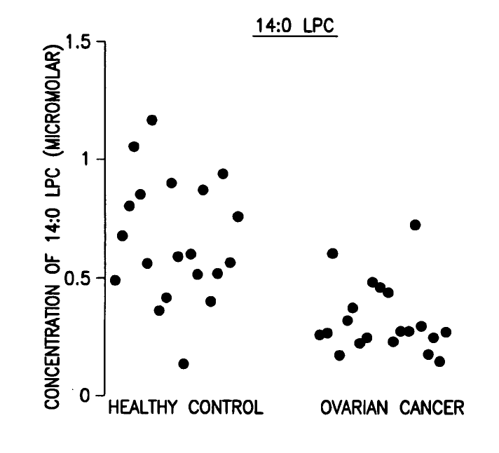

### CROSS-REFERENCE TO RELATED APPLICATIONS

This application claims the benefit under 35 USC 119(e) of U.S. provisional application Ser. No. 61/002,282 filed Nov. 7, 2007, U.S. provisional application Ser. No. 61/002,989 filed Nov. 14, 2007 and U.S. provisional application Ser. No. 61/066,331 filed Feb. 20, 2008, the entire contents of all of which provisional applications are incorporated by reference herein.

### BACKGROUND OF THE INVENTION

1. Field of the Invention

Methods for detecting a cancer, such as ovarian cancer, are disclosed herein. Also discussed herein are methods for monitoring a cancer, such as ovarian cancer. More particularly, disclosed herein are methods for detecting ovarian cancer in a test subject by determining the amount of a lysophosphatidyl choline (“LPC”) in a sample of a bodily fluid taken from the test subject. The methods discussed herein are particularly useful as a screening test for ovarian cancer.

2. Background Information

Ovarian cancer is one of the deadliest cancers for women, due to its high fatality rate. In the United States in 2007, it was estimated that 22,430 women would be diagnosed with ovarian cancer and 15,280 women would die of ovarian cancer. Unfortunately, heretofore, only 25% of ovarian cancer patients were diagnosed at stage I. Most of the patients were diagnosed at an advanced stage, stage III or IV, at which the 5-year survival rate decreases to 20 to 25% from 95% at stage I.

Presently, the most commonly used biomarker for diagnosing ovarian cancer is CA-125, a group of surface glycoproteins with uncertain biological function. Although CA-125 is elevated in 82% of women with advanced ovarian cancer, it has very limited clinical application for the detection of early stage disease, exhibiting a positive predictive value of less than 10%. The addition of physical examination by diagnostic ultrasound improves the positive predictive value to 20%, which is still too low to meet the requirement for cancer detection. Developing a clinical test to diagnose ovarian cancer with high sensitivity and specificity at the early stage has become the most urgent issue in battling this refractory disease.

Frequently, the detection of cancer depends upon the detection and inspection of a tumor mass, which has reached sufficient size to be detected by physical examination. The detection of molecular markers of carcinogenesis and tumor growth can solve many of the problems associated with the physical examination of tumors. Samples taken from the patient for screening by molecular techniques are typically blood or urine, and thus require minimally invasive techniques. Thus, they can be used on a regular basis to screen for cancers. In addition, because molecular markers may appear before the tumor reaches a detectable size, it is possible to detect cancers at very early stages in the progression of the disease.

Biomarkers identified from serum proteomic analysis for the detection of ovarian cancer are discussed in Z. Zhang et al., Cancer Research, 64, 5882-5890, Aug. 15, 2004.

Methods for detecting a cancer associated with elevated concentrations of lysophospholipids have been described in US 2002/0123084 and US 2002/0150955.

U.S. Pat. No. 6,500,633 discloses a method of detecting carcinomas by measuring the level of a glycerol compound, such as glycerol-3-phosphate, in a plasma, serum or urine specimen from a patient.

US 2007/0196875 (inventors: Lian Shan and Stanley L. Hazen) discloses a method for detecting ovarian cancer using plasmenyl-PA as a marker.

US 2008/0020472 (inventors: Lian Shan and Lorelei D. Davis) discloses a method for detecting ovarian cancer using plasmenyl-PE as a marker.

### SUMMARY. OF THE INVENTION

It is an object of the present invention to provide a non-invasive method for detecting a cancer, such as ovarian cancer, in a test subject.

It is another object of the present invention to utilize a molecular marker for the screening and diagnosis of a cancer, such as ovarian cancer.

It is a further object of the present invention to provide a non-invasive method to monitor the presence of a cancer, such as ovarian cancer, over time.

The above objects, as well as other objects, advantages and aims, are satisfied by the present invention.

The present invention concerns a method of detecting a cancer (for example, ovarian cancer) in a test subject comprising:

(a) determining the amount of a lysophosphatidyl choline in a sample of a bodily fluid taken from the test subject, and

(b) comparing the amount of the lysophosphatidyl choline in the sample of the bodily fluid taken from the test subject to a range of amounts found in samples of the bodily fluid taken from a group of normal subjects of the same species as the test subject and lacking the cancer (for example, if the bodily fluid taken from the test subject is serum, then the bodily fluid taken from each member of the group of normal subjects will also be serum), whereby a change in the amount (such as a lower amount) of the lysophosphatidyl choline in the sample of the bodily fluid taken from the test subject indicates the presence of the cancer (for example, ovarian cancer).

The present invention further concerns a method for monitoring a cancer (for example, ovarian cancer) in a test subject over time comprising:

(a) determining the amount of a lysophosphatidyl choline in a sample of a bodily fluid taken from the test subject at a first time,

(b) determining the amount of the lysophosphatidyl choline in a sample of the bodily fluid taken from the test subject at a second time (for example, if the bodily fluid in step (a) is serum, then the bodily fluid in step (b) will also be serum), which is later than the first time,

(c) comparing the amounts of the lysophosphatidyl choline in each of step (a) and step (b) to determine whether there has been an increase or a decrease in the amount of the lysophosphatidyl choline in a sample of the bodily fluid taken from the test subject at the later time relative to the amount of the lysophosphatidyl choline in the sample of the bodily fluid taken from the test subject at the first time, whereby a decrease from the amount of the lysophosphatidyl choline in the sample of the bodily fluid taken from the test subject at the later time indicates the presence of, or worsening of, the cancer (for example, ovarian cancer), or an increase from the amount of the lysophosphatidyl choline in the sample of the bodily fluid taken from the test subject at the later time indicates an absence, or improvement of, the cancer (for example, ovarian cancer).

### BRIEF DESCRIPTION OF THE DRAWING

The sole FIGURE is a graph showing the levels of 14:0 lysophosphatidyl choline (“14:0 LPC”) in plasma samples from ovarian cancer patients and patients without ovarian cancer (“healthy controls”).

### DETAILED DESCRIPTION OF THE INVENTION

Applicants have discovered that the lipid lysophosphatidyl choline (“LPC”) can be used in the methods disclosed herein for detecting a cancer, such as ovarian cancer, and monitoring a cancer, such as ovarian cancer, in a test subject.

A non-limiting example of the lysophosphatidyl choline that can be used in the methods disclosed herein is 14:0 LPC.

The molecule weight, chemical name and structure for 14:0 are as follows:

14:0 LPC

mw 467.58

1-Myristoyl-2-Hydroxy-sn-Glycero-3-Phosphocholine

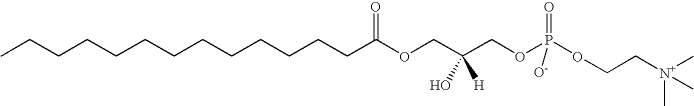

In, an embodiment of the invention, an amount of a lysophosphatidyl choline (“LPC”) found in a sample of a bodily fluid taken from a test subject is compared to the amount of the LPC found in samples taken from normal subjects of the same species as the test subject lacking a cancer (for example, ovarian cancer) (e.g., if the test subject is a human, then the normal subject is a human who does not have the cancer (for example, ovarian cancer)). Thus, the amount of a LPC taken from a test subject, e.g., a female, is determined, and a range of amounts of the LPC taken from normal females, e.g., lacking ovarian cancer, is obtained. A lower amount of the LPC found in the sample of the bodily fluid taken from the test subject when compared to a range of amounts of the LPC in samples of the bodily fluid taken from a group of normal subjects of the same species as the test subject and lacking the cancer (for example, ovarian cancer), indicates the presence of the cancer (for example, ovarian cancer).

The amount of the LPC detected in the sample taken from a test subject may be measured by first extracting lipids as described in detail infra. The amount of the LPC is then quantified using standard procedures, such as mass spectroscopy, gas chromatography, HPLC, NMR or other approaches.

In addition to the direct measurement of the LPC by extraction, antibodies, such as monoclonal antibodies reactive with the LPC can be used in an assay to detect the amount of the LPC. For example, anti-LPC antibodies may be labeled using standard procedures and used in assays including radioimmunoassay (RIA), both solid and liquid phase, fluorescence-linked assays or enzyme-linked immunosorbent assays (ELISA), wherein the antibody is used to detect the presence and amount of the LPC.

The test subject can be a eukaryotic organism, preferably a vertebrate, including, but not limited to, a mammal, a bird, a fish, an amphibian or a reptile. Preferably, the subject is a mammal, most preferably a human. The bodily fluid includes, but is not limited to, plasma, serum, urine, saliva, ascites, cerebral spinal fluid or pleural fluid. Preferably, the bodily fluid is plasma or a serum which is obtained from a whole blood specimen from the test subject.

The methods disclosed herein can be used to detect, screen or monitor for a broad range of cancers at an early stage. Such cancers include gynecological cancers, including ovarian cancer, breast cancer, cervical cancer, uterine cancer, endometrial cancer, peritoneal cancer, fallopian tube cancer and vulva cancer. Other cancers that can be detected, screened or monitored according to the methods disclosed herein include, but are not limited to, testicular cancer, colon cancer, lung cancer, prostate cancer, bladder cancer, kidney cancer, thyroid cancer, stomach cancer, pancreatic cancer, brain cancer, liver cancer, ureter cancer, esophageal cancer and larynx cancer. The methods disclosed herein are preferably directed to detecting ovarian cancer.

The methods disclosed herein are non-invasive and require only a bodily fluid specimen, such as a blood specimen from the test subject (patient). Thus, such methods are particularly useful for screening patients who have not been previously diagnosed as having ovarian cancer. Such patients include women at elevated risk by virtue of a family history of the disease, premenopausal women with anovulatory cycles and postmenopausal women. The methods disclosed herein include a screening test for identifying within a risk population, a subject population with a greater propensity for developing ovarian cancer.

The methods disclosed herein can provide a number of benefits. First, the methods provide a rapid and economical screen for large numbers of subjects to promote early diagnosis of ovarian cancer, which can result in improved quality of life and better survival rates for patients.

Using the methods disclosed herein for prognosis, the medical professional can determine whether a subject having ovarian cancer in the early stages requires therapy or does not require therapy. This could also identify subjects who may not benefit from a particular form of therapy, e.g., surgery, chemotherapy, radiation or biological therapies. Such information could result in an improved therapy design for obtaining better responses to therapy.

The methods disclosed herein can also be used to identify patients for whom therapy should be altered from one therapeutic agent to another. This could obviate the need for “second look” invasive procedures to determine the patient's response to the therapy and facilitate decisions as to whether the particular type of therapy should be continued, terminated or altered.

Because cancers may recur in a significant number of patients with advanced cancers, early detection and continued monitoring over time using the methods disclosed herein can identify early occult (i.e., “hidden”) recurrences prior to symptoms presenting themselves.

In addition, methods disclosed herein will facilitate distinguishing benign from malignant tumors. Masses in the ovary can be initially detected using procedures such as ultrasound or by physical examination. Thereafter, the methods disclosed herein can be used to diagnose the presence of a cancer (for example, ovarian cancer). This could obviate the need for surgical intervention, and/or identify subjects for whom continued monitoring is appropriate, resulting in improved early detection and survival for ovarian cancer patients.

### EXAMPLES

The present invention will now be described in the context of the following non-limiting examples.

### Example 1

### Quantitative Determination of 14:0 LPC in Human Plasma

(a). Extraction of 14:0 LPC from Human Plasma

14:0 LPC in plasma was extracted using a modified Bligh-Dyer method, which follows the following procedure: First mix 400 pmol heavy isotope-labeled [¹³C₃] 18:0 LPC with 50 μl plasma. The mixture was vortexed and 2 ml 2:1 (v:v) methanol-chloroform was added. The mixture was vortexed again and kept at room temperature for 10 minutes. Then it was centrifuged at 4000 rpm at 10° C. for 10 minutes. The top liquid layer was transferred into another tube and dried under nitrogen. The dried pellet was dissolved in 400 μl 100 mM ammonium acetate in methanol and centrifuged at 9000 rpm for 5 minutes. The supernatant was further diluted by 1:9 ratio with 360 μl 100 mM ammonium acetate in methanol. 30 μl of the mixture was then injected into the LC/ESI/MS/MS system.

(b) LC/ESI/MS/MS Analysis of 14:0 LPC

LC/ESI/MS/MS analysis of 14:0 LPC was performed using a Quattro Micro mass spectrometer (Micromass, Altrincham, U.K.) equipped with an electrospray ionization (ESI) probe and interfaced with a Shimadzu SCL-10Avp HPLC system (Shimadzu, Tokyo. Japan). Lipids were separated with a Betabasic-18 column (20×2.1 mm, 5 μm, Thermo Electron, Waltham, Mass.), protected by a Betabasic 18 pre-column (10×2.1 mm, 5 μm, Thermo Electron, Waltham, Mass.). 0.2% formic acid aqueous solution was used as mobile phase A and 0.2% formic acid in methanol was used as mobile phase B. The flow rate was 200 μl/minutes. The gradient used was as follows: the column was first equilibrated with 50% B (50% A), followed by a linear change from 50% B (50% A) to 100% B (0% A) in the first 4 minutes. The gradient was kept at 100% B in the following 8 min. In the following 4 minutes, the gradient was changed back to 50% B (50% A) to re-equilibrate the column. Mass spectrometric analyses were performed online using electrospray ionization tandem mass spectrometry in the positive multiple reaction monitoring (MRM) mode. The MS parameters are: capillary voltage, 3.0 KV; cone voltage, 50 V; source temperature, 100° C.; desolvation temperature, 350° C.; flow rate of desolvation gas, 500 L/hr; flow rate of cone gas, 50 L/hr; mass resolution of both parent and daughter ions, 15.0; multiplier, 650. The MRM transitions used to detect 14:0 LPC were the mass to charge ratio (m/z) for their molecular cation M⁺, 468 and their corresponding daughter ion, 184 (collision energy 22 eV).

### Example 2

### Data Analysis

Data analysis was done using the student t-test and the peak area ratio of analyte to internal standard was determined. The results are shown in the FIGURE.

Forty (40) plasma samples were collected. Among them were twenty (20) stage III ovarian cancer patients and twenty (20) healthy controls.

The 14:0 LPC data are expressed as concentration in μM. The results are shown in Table 1 below and in the FIGURE.

TABLE 1
Levels of 14:0 LPC, its corresponding standard deviation, and
p value of ovarian cancer patients related to healthy controls

| | Ovarian Cancer | | Healthy control | | |
|---|---|---|---|---|---|
| | LPC | Standard | LPC | Standard | |
| | level | Deviation | level | Deviation | p value |
| 14:0 LPC | 0.314 | 0.152 | 0.656 | 0.255 | <0.0001 |

What is claimed is:

1. A method of detecting a cancer in a test subject comprising:
(a) determining the amount of a lysophosphatidyl choline in a sample of a bodily fluid taken from a test subject, and
(b) comparing the amount of the lysophosphatidyl choline in the sample of the bodily fluid from the test subject to a range of amounts of the lysophosphatidyl choline found in samples of said bodily fluid taken from a group of normal subjects of the same species as the test subject and lacking the cancer, whereby a change in the amount of the lysophosphatidyl choline in the sample of the bodily fluid from the test subject indicates the presence of the cancer.

2. The method of claim 1, wherein the test subject is a human.

3. The method of claim 2, wherein the cancer is ovarian cancer.

4. The method of claim 3, wherein, in step (b), the change in the amount is a lower amount.

5. The method of claim 4, wherein the bodily fluid is plasma.

6. The method of claim 5, wherein the lysophosphatidyl choline is 14:0 LPC.

7. A method for monitoring a cancer in a test subject over time comprising:
(a) determining the amount of a lysophosphatidyl choline in a sample of a bodily fluid taken from a test subject at a first time,
(b) determining the amount of the lysophosphatidyl choline in a sample of the bodily fluid taken from said test subject at a second time, which is later than the first time,
(c) comparing the amounts of the lysophosphatidyl choline in each of step (a) and step (b) to determine whether there has been an increase or a decrease in the amount of the lysophosphatidyl choline in the sample of the bodily fluid taken from the test subject at the later time relative to the amount of the lysophosphatidyl choline in the sample of the bodily fluid taken from the test subject at the first time, whereby a decrease from the amount of the lysophosphatidyl choline in the sample of the bodily fluid taken from the test subject at the later time indicates the presence of or worsening of the cancer, or an increase in the amount of the lysophosphatidyl choline in the sample of the bodily fluid taken from the test subject at the later time indicates an absence or improvement of the cancer.

8. The method of claim 7, wherein the test subject is a human.

9. The method of claim 8, wherein the cancer is ovarian cancer.

10. The method of claim 9, wherein the lysophosphatidyl choline is 14:0 LPC.
